(19) United States
(12) Patent Application Publication
(10) Pub. No.: US 2008/0194903 A1
(43) Pub. Date: Aug. 14, 2008

(54) Metathesis Method for Purifying Starting Products

(75) Inventors: Markus Schubert, Ludwigshafen (DE); Jurgen Stephan, Mannheim (DE); Frank Poplow, Ludwigshafen (DE); Thomas Heidemann, Viernheim (DE); Uwe Diehlmann, Hassloch (DE); Michaela Maltry, Bobenheim-Roxheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigsshafen (DE)

(21) Appl. No.: 11/817,237

(22) PCT Filed: Feb. 24, 2006
(86) PCT No.: PCT/EP06/60276

§ 371 Date: Aug. 28, 2007

(30) Foreign Application Priority Data
Feb. 28, 2005 (DE) 102005009596.8

(51) Int. Cl. C07C 7/12

(52) U.S. Cl. 585/823

(57) ABSTRACT

Processes comprising: (a) providing a feed stream comprising a C₄ compound having at least a nonaromatic C—C double bond or C—C triple bond; (b) contacting the feed stream with an adsorbent which has been activated at a temperature of 450 to 1000° C. comprising at least 3% by weight of aluminum oxide to remove one or more impurities from the feed stream; and (c) contacting the feed stream from which one or more impurities have been removed with a metathesis catalyst under metathesis reaction conditions to form a compound or a mixture of compounds having a nonaromatic C—C double bond or C—C triple bond.

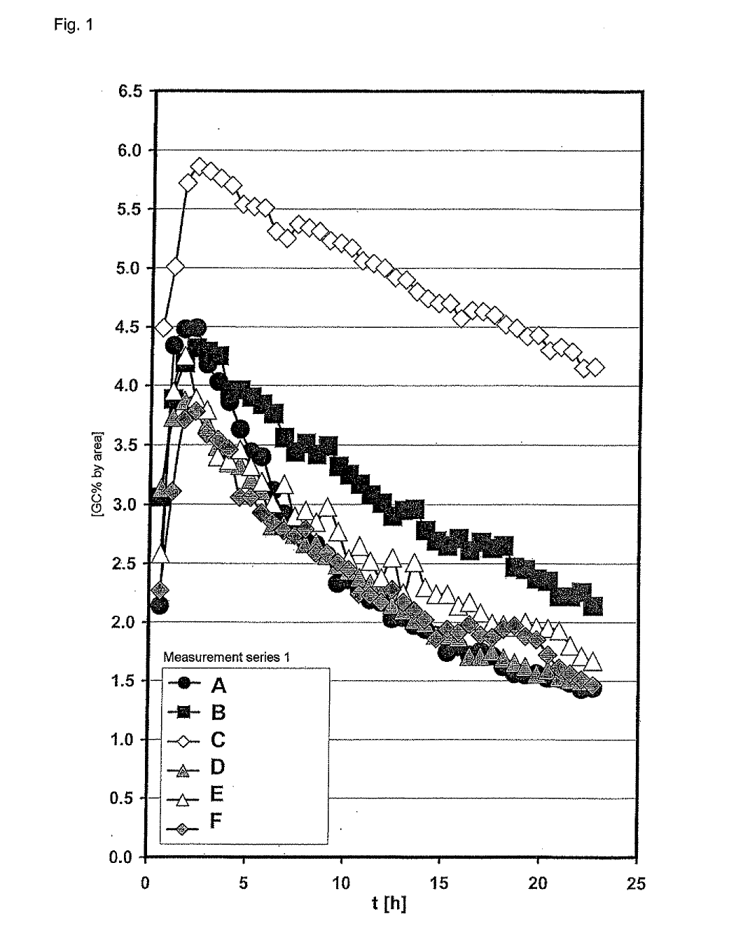

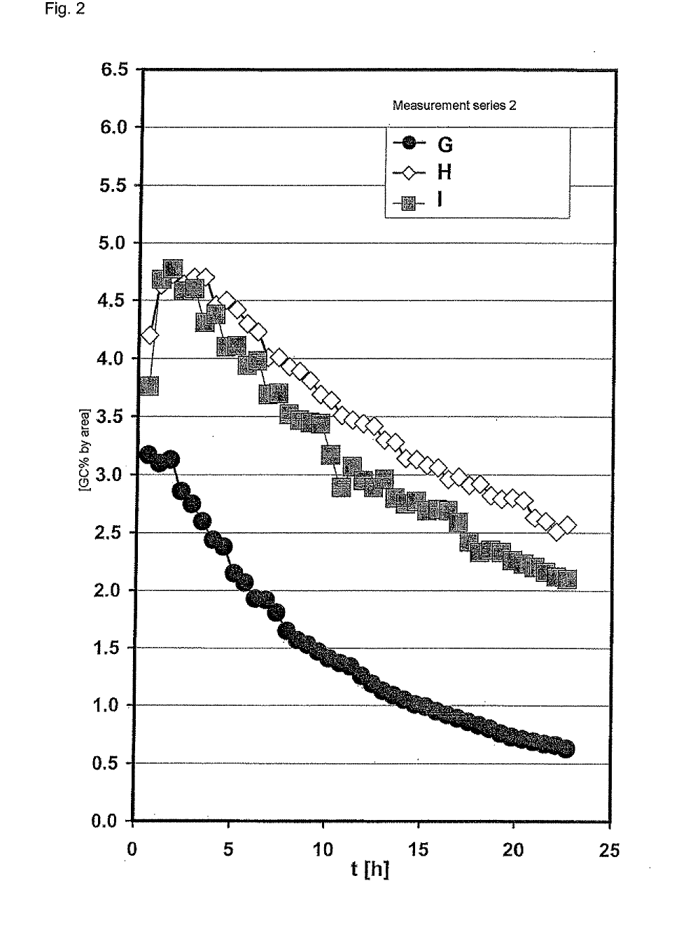

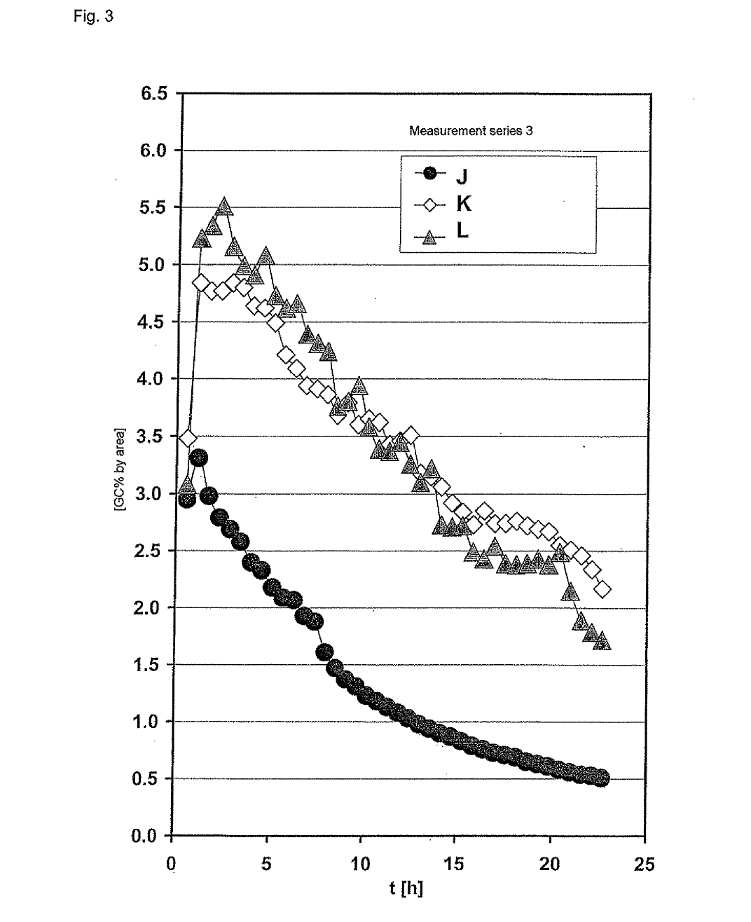

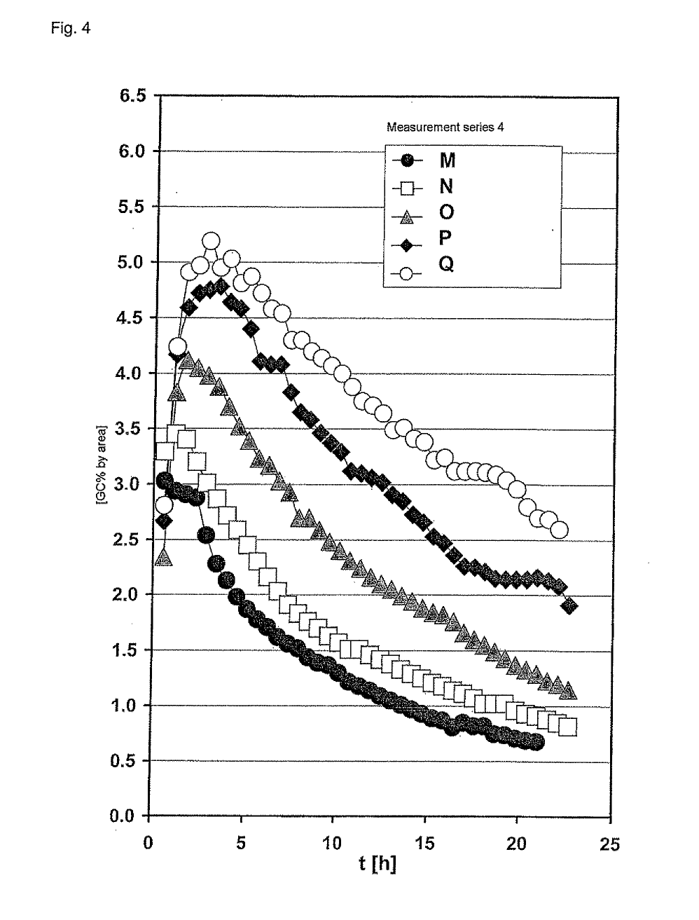

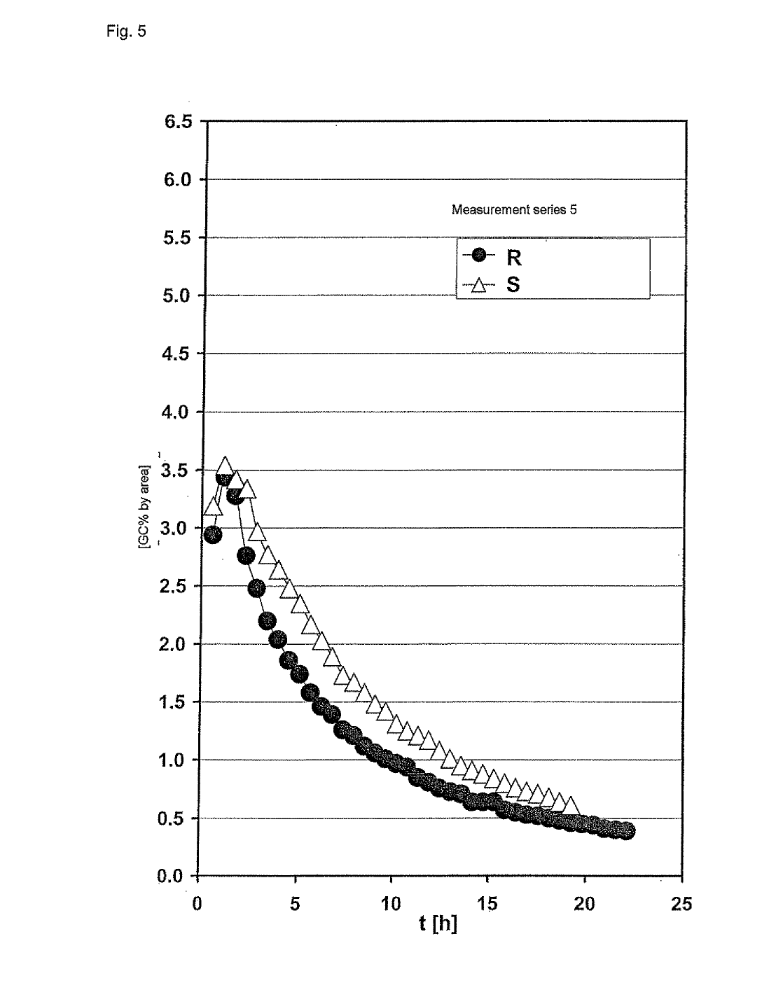

The present invention relates to a process for preparing a compound or a mixture of compounds having a nonaromatic C—C double bond or C—C triple bond by metathesis and prior purification of a compound or a mixture of compounds having a nonaromatic C—C double bond or C—C triple bond.

The metathesis of nonaromatic unsaturated hydrocarbon compounds is a long-established method of breaking and reforming C—C bonds (e.g. Mol, J. C., Chapt. 4.12.2 “Alkene Metathesis” in “Handbook of Heterogeneous Catalysis”, Eds. Ertl, G., Knozinger, H., Weitkamp, J., VCH, Weinheim 1997; Weissermehl, K., Arpe, H.-J., Chapt. 3.4 “Olefin-Metathese” in “Industrielle Organische Chemie”, 4th Edition, VCH, Weinheim 1994). Various types of catalysts have been described for a heterogeneously catalyzed metathesis. In the temperature range up to about 120° C., the use of supported Re₂O₇ or Re(CO)₁₀ catalysts is customary (Mol, J. C., Chapt. 4.12.2 “Alkene Metathesis” in “Handbook of Heterogeneous Catalysis”, Eds. Ertl, G., Knözinger, H., Weitkamp, J., VCH, Weinheim 1997). At somewhat higher temperatures of up to 400° C., it is possible to employ, according to the literature, catalysts based on MoO₃, CoO—MoO₃, MoS₂, Mo(CO)₆ or various supported Mo complexes, while at higher temperatures of up to 540° C. it is also possible to use systems based on WO₃, WS₂, W(CO)₆ or supported W complexes (Molt, J. C., Chapt. 4.12.2 “Alkene Metathesis” in “Handbook of Heterogeneous Catalysis”, Eds. Ertl, G., Knözinger, H., Weitkamp, J., VCH, Weinheim 1997; Weissermehl, K., Arpe, H.-J., Chapt. 3.4 “Olefin-Metathese” in “Industrielle Organische Chemie”, 4th Edition, VCH, Weinheim 1994; Heckelsberg, L. F., Banks, R. L., Bailey, G. C., Ind. Eng. Chem. Prod. Res. Develop. 8 (1969), 259-261). As an alternative, the reaction can in principle also be carried out over homogeneous catalysts, usually Ru, Mo or W complexes (Grubbs, Robert, H. (editor), Handbook of Metathesis, 1st Edition, August 2003—ISBN—3-527-30616-1—Wiley-VCH, Weinheim)

It is known to those skilled in the art that metathesis catalysts are very sensitive to impurities (feed poisons) in the feed stream (Weissermehl, K., Arpe, H.-J., Chapt. 3.4 “Olefin-Metathese” in “Industrielle Organische Chemie”, 4th Edition, VCH, Weinheim 1994). Such feed poisons are, for example, strongly polar or protic compounds such as N—, O—, S— and halogen-comprising components (typical examples are water, alcohols, ethers, ketones, aidehydes, acids, acid derivatives, amines, nitriles, thiols), acetylenes or dienes, in particular allenes. The consequences are reduced activity and severely shortened cycle times or lives of the metathesis catalysts used.

Various techniques can be used for removing the feed poisons. Part of the compounds can be converted by chemical reaction into noncritical components. For example, acetylenes and diolefins can be largely removed from the monoolefin stream in a selective hydrogenation (Weissermehl, K., Arpe, H.-J., Chapt. 3.4 Olefin-Metathese” in “Industrielle Organische Chemie”, 4th Edition, VCH, Weinheim 1994). Heteroatom-comprising components in particular are preferably removed from the feed stream by adsorption. Thus, U.S. Pat. No. 3,915,897 describes, for example, a combination of calcium hydride, 13X molecular sieves and magnesium oxide for purifying a C₄-olefin stream. EP 1,280,749 describes a process for preparing alcohols with adsorptive removal of P-comprising impurities and dienes from an olefin mixture (C₆ to C₃₆) over zeolites, aluminum oxides or activated carbon.

These adsorbents usually have to be activated before use by heating at temperatures of 200-250° C. in a stream of inert gas in order to desorb water and CO₂ which have been adsorbed during storage. Only alkaline earth metal oxides such as MgO are brought to significantly higher temperatures beforehand to decompose carbonates which have been formed on the surface. The industrially customary regeneration of the adsorbent (X) is likewise effected by desorption at temperatures of 200-250° C. (“thermal swing adsorption”), and in some cases simply by depressurization (“pressure swing adsorption”) (“Sylobead” brochure from Grace GmbH & Co. KG, In der Hollerhecke 1, 67545 Worms/Germany). For use in specific chemical processes, regenerations under more drastic conditions have also been described in some cases. Thus, for instance, DE 198,45,857 describes a process for the oligomerization of monoolefins in which the adsorbent is regenerated at temperatures of up to 800° C., preferably in an oxidative atmosphere, EP 1,280,749 describes a process for preparing alcohols, in which the bed of adsorbent is regenerated in an oxygen-comprising atmosphere at temperatures of from 200 to 600° C.

In the case of the metathesis of olefinic C₄ fractions over Re-comprising catalysts, adsorptive purification of the feed stream is in principle prior art. Thus, for instance, DE 10013253 mentions molecular sieves and high surface area aluminum oxides as adsorbents which are suitable in principle. DE-A-10309070 mentions, in particular, molecular sieves, for example 3 Ø or 13X, as preferred adsorbents for the starting materials for such a C₄-olefin metathesis. Oxidative treatment at temperatures of from 100 to 350° C. is said to be a suitable regeneration procedure for the molecular sieves.

It was an object of the present invention to provide an economical process for the metathesis of hydrocarbons having at least one nonaromatic C—C multiple bond.

We have accordingly found a process for preparing a compound or a mixture of compounds having a nonaromatic C—C double bond or C—C triple bond (compound A) from another compound or a mixture of other compounds having a nonaromatic C—C double bond or C—C triple bond (compound B) by
I. in step (I) freeing the compound (B) of impurities by bringing it into contact with an adsorbent which comprises at least 3% by weight of aluminum oxide and has been activated at temperatures of from 450 to 1000° C. (adsorbent X) and
II. in step (II), bringing the compound B which has been freed of impurities in step (I) into contact with a metathesis catalyst under conditions customary for metathesis reactions.

The compound (A) is preferably propene, 3-hexene, ethylene or 2-pentene or a mixture thereof. To prepare it, a C₄ starting compound such as 1-butene, 2-butene or ethylene or a mixture thereof is preferably used as compound (B). Compound (B) particularly preferably comprises butenes and, if appropriate, additionally ethylene, with the butenes being used in the form of a mixture with butanes.

However, further possible compounds (B) are unsaturated esters, nitrites, ketones, aidehydes, acids or ethers, as is described, for example, in Xiaoding, X., Imhoff, P., von den Aardweg, C. N., and Mol, J. C., J. Chem. Soc., Chem. Comm. (1985), p. 273.

The abovementioned C₄ starting compounds are usually made available in the form of a raffinate II. A raffinate II is a C₄ fraction which generally has a butene content of from 30 to 100% by weight, preferably from 40 to 98% by weight. Apart from the butenes, saturated C₄-alkane in particular can be additionally present. The way of obtaining such raffinates II is generally known and is described, for example, in EP-A-1134271.

In particular, 1-butene-comprising olefin mixtures or 1-butene which is obtained by distilling off a 1-butene-rich fraction from raffinate II are used. 1-Butene can likewise be obtained from the remaining 2-butene-rich fraction by subjecting the 2-butene-rich fraction to an isomerization reaction and subsequently separating the product into a 1-butene-rich fraction and a 2-butene-rich fraction by distillation. This process is described in DE-A-10311139.

Propene or a mixture of propene and 3-hexene can be particularly advantageously prepared according to the process of the invention by metathesis of a mixture comprising 2-butene and ethylene or 1-butene and 2-butenes, and 3-hexene and ethylene can be prepared by metathesis of 1-butene. Corresponding processes are described in detail in DE-A-19813720, EP-A-1134271, WO 021083609, DE-A-10143160.

In general, the compound (A) is prepared continuously by subjecting a stream comprising the compound (B) (stream B) to the steps (I) and (II).

The process is usually carried out continuously by making the compound (B) available in the form of a stream comprising compound (B) (stream B) and passing this continuously in accordance with step (I) through a guard bed which comprises adsorbent (X) and is installed in a reactor (guard bed X) to give a purified stream (B) and subsequently passing this continuously in accordance with step II through a catalyst bed which comprises a metathesis catalyst and is installed in a reactor to give compound (B).

Stream (B) is preferably a C₄-hydrocarbon stream (hereinafter also referred to as “C₄ feed stream”).

In one process variant, the C₄ feed stream is made available by
Ia) in step (Ia), subjecting naphtha or other hydrocarbon compounds to a steam cracking or FCC process and taking off a C₄-olefin mixture comprising 1-butene, 2-butene and more than 1000 ppm by weight of butadienes and possibly butynes and possibly isobutene from the stream formed in the cracking process and

IIa) preparing a C₄-hydrocarbon stream consisting essentially of 1-butene, 2-butenes and possibly butanes and possibly isobutene (raffinate I) from the C₄-olefin mixture formed in step (Ia) by hydrogenating the butadienes and butynes to butenes or butanes by means of selective hydrogenation or removing the butadienes and butynes by extractive distillation to such an extent that the 1,3-butadiene content is not more than 1000 ppm by weight.

In another process variant, the C₄ feed stream is made available by
Ib) in step (Ib), preparing a C₄-olefin mixture comprising 1-butene, 2-butenes and more than 1000 ppm by weight of butadienes and possibly butynes and possibly butanes from a hydrocarbon stream comprising butanes by dehydrogenation and subsequent purification,
IIb) preparing a C₄-hydrocarbon stream consisting essentially of isobutene, 1-butene, 2-butenes and possibly butanes (raffinate I) from the C₄-olefin mixture formed in step (Ib) by hydrogenating the butadienes and butynes to butenes or butanes by means of selective hydrogenation or removing the butadienes and butynes by extractive distillation to such an extent that the 1,3-butadiene content is not more than 1000 ppm by weight.

In the generally known FCC process (cf. Ullmann's Encyclopedia of Industrial Chemistry, Wiley-VCH Verlag GmbH, Weinheim, Germany, Sixth Edition, 2000 Electronic Release, Chapter Oil Refining, 3.2. Catalytic Cracking), the appropriate hydrocarbon is vaporized and brought into contact in the gas phase with a catalyst at a temperature of from 450 to 500° C. The particulate catalyst is fluidized by means of the hydrocarbon stream conveyed in countercurrent. Catalysts employed are usually synthetic crystalline zeolites.

In the likewise generally known steam cracking process (cf. A. Chauvel, G. Lefebvre: Petro-chemical Processes, 1 Synthesis—Gas Derivatives and Major Hydrocarbons, 1989 Editions Technip 27 Rue Ginoux 75737 Paris, France, Chapter 2), the hydrocarbon is mixed with steam and, depending on the residence time, heated to temperatures of from 700 to 1200° C. in tube reactors and then cooled rapidly and separated into individual fractions by distillation.

If the 1,3-butadiene content of the C₄-olefin mixture obtained in step (Ia) or step (Ib) is 5% by weight or more, preference is given to reducing the 1,3-butadiene content to a content of from 1000 ppm by weight to 5% by weight by means of extractive distillation and subsequently reducing the 1,3-butadiene content further to 1000 ppm by weight or less by means of selective hydrogenation.

Compounds (B) which are made available by means of these or other industrial processes frequently comprise a compound from the group consisting of water, alcohols, ethers, ketones, aldehydes, acids, in particular carboxylic acids, acid derivatives, amines, nitrites, thiols, acetylenes and dienes, in particular allenes, as impurity. The total amount of feed poisons present in the compounds (B) is typically from 1 to 1000 ppm by weight.

The adsorbent (X) used in step (I) preferably comprises at least 10% by weight, particularly preferably at least 75% by weight, of aluminum oxide. The aluminum oxide comprised in adsorbent (X) is preferably present in a phase selected from the group consisting of gamma-Al₂O₃, delta-Al₂O₃, theta-Al₂O₃, and eta-Al₂O₃, or a hydrated precursor of one of these phases. The hydrated precursor of one of these phases is, for example, Boehmite, pseudo-boehmite or hydrargillite. The adsorbent (X) is very particularly preferably pure gamma-aluminum oxide.

In addition, the adsorbent (X) can further comprise auxiliaries or further adsorption-active compounds, for example aluminosilicates, aluminum phosphates or alkaline metal oxides, alkaline earth metal oxides or SiO₂, preferably aluminosilicates, aluminum phosphates or alkaline earth metal oxides or SiO₂.

The adsorbent (X) is advantageously brought into contact with an inorganic mineral acid, for example H₂SO₄, HCl, HClO₄, HNO₃, H₃PO₄, and the mineral acid is subsequently removed again before the adsorbent first comes into contact with compound (B).

The activation of the adsorbent (X) before it first comes into contact with compound (B) will hereinafter also be referred to as “first activation”.

Furthermore, it has been found to be useful to bring the adsorbent (X) into contact with a compound or a mixture of compounds comprising at least one of the elements W, Mo, Zr, Ti, Hf, Si, P, Fe, Nb, Ta, Mn and V prior to the first activation. These are preferably oxides or phosphates. Precursors of these compounds, i.e compounds which are converted into the compounds mentioned during the activation, are also suitable.

Likewise, the basicity of the adsorbent can be increased if necessary, for example by doping with zinc compounds, alkali metal compounds or alkaline earth metal compounds or compounds of the lanthanide elements, e.g. their hydroxides or oxides, in an amount of preferably from 100 to 1000 ppm by weight.

The adsorbent (X) preferably has a surface area of at least 50 m²/g, preferably more than 100 m²/g, and a pore volume of at least 0.3 ml/g, preferably more than 0.4 ml/g. The surface area is determined by the method of Stephen Brunauer, Paul Emmett and Edward Teller in accordance with DIN 66131. The pore volume is determined by Hg porosimetry in accordance with DIN 66133.

The adsorbent (X) is usually used as a fixed bed and is present as shaped bodies, for example spheres, extrudates or granules.

The bringing into contact of the adsorbent (X) with the compound (B) will hereinafter also be referred to as “adsorption”.

The activation is usually effected by bringing the adsorbent (X) into contact with a gas which has a temperature of from 450 to 1000° C., preferably from 500 to 900° C., very particularly preferably from 550 to 850° C. The adsorbent (X) and the gas are preferably brought into contact by passing the gas through the fixed bed (X).

Gases suitable for the activation are oxygen, carbon dioxide, air, nitrogen, natural gas or mixtures thereof.

The activation is preferably carried out until the weight of the adsorbent (X) is no longer decreased by the activation and virtually no carbon or no carbon-comprising compounds is/are adsorbed on the adsorbent (X). The absence of carbon or carbon-comprising compounds can be checked in a simple manner by means of elemental analysis.

The adsorption is preferably carried out at temperatures of from 0 to 150° C., particularly preferably from 20 to 110° C., in particular in the range from 20 to 50° C. The adsorption is usually carried out at pressures of from 2 to 100 bar, preferably from 5 to 50 bar. The stream (B) is preferably passed as a liquid phase over the guard bed (X).

The time between activation and adsorption is preferably less than 10 days, particularly preferably less than 5 days and very particularly preferably less than 1 day.

After activation and before the adsorption, care has to be taken to ensure that the adsorbent (X) no longer comes into contact with a gas atmosphere which comprises more than 1000ppm by volume of a gas selected from the group consisting of carbon dioxide and watervapor.

The adsorbent (X) is usually not ready for use immediately but is advantageously activated to attain its full capacity before the adsorbent (X) is first brought into contact with the compound (B), i.e. before the first use.

In general, it will be necessary to carry out the activation of the adsorbent (X) not only before the first use but also after particular periods of adsorption. The activation of an adsorbent (X) or guard bed (X) which has been brought into contact for a particular period of time with a compound (B) will hereinafter also be referred to as “regeneration”. Regeneration is necessary at the latest when the impurities are no longer adsorbed by the adsorbent (X) because its capacity is exhausted. In general, regeneration is necessary after a period of from 1 hour to 4 months.

Particularly when relatively high molecular weight carbon-comprising compounds have been adsorbed on the adsorbent (X), it is advisable to carry out the activation using an oxygen-comprising gas stream. In this activation, the carbon-comprising compounds are oxidized to carbon dioxide and removed. The regeneration is in this case preferably carried out by interrupting the bringing into contact of the guard bed (X) with compound (B) and passing an inert gas stream through the guard bed (X) at a temperature of from 0 to 450° C. and, if appropriate, subsequently passing an oxygen-comprising gas stream through the guard bed (X) at a temperature of from 450 to 700° C.

Possible oxygen-comprising gas streams are gas streams which comprise, in addition to the abovementioned constituents of the inert gas stream, from 0.05 to 20% by weight of oxygen.

The metathesis reaction in step (II) is not critical and can be carried out in a customary fashion (cf., for example, Mol, J. C., Chapt. 4.12.2 “Alkene Metathesis” in “Handbook of Heterogeneous Catalysis”, Eds. Ertil G., Knözinger, H., Weitkamp, J., VCH, Weinheim 1997; Weissermehl, K., Arpe, H.-J., Chapt. 3.4 “Olefin-Metathese” in “Industrielle Organische Chemie”, 4th Edition, VCH, Weinheim 1994)

As metathesis catalyst in step (II), preference is given to using a metathesis catalyst which comprises at least one compound comprising at least one element selected from the group consisting of Re, W and Mo.

In step (II), preference is given to using a metathesis catalyst which comprises rhenium oxide on aluminum oxide and carrying out the metathesis reaction in the liquid phase at a temperature of from 0 to 120° C.

Particular preference is given to catalysts having a content of at least 0.3% by weight of Re atoms, very particularly preferably a content of at least 1% by weight of Re atoms. Usual reaction temperatures over Re-comprising catalysts are from 0 to 150° C., preferably from 20 to 110° C. Usual reaction pressures are from 2 to 100 bar, preferably from 5 to 50 bar, particularly preferably from 20 to 40 bar.

In the reaction of substituted olefins, use is frequently made of a cocatalyst, for example a tin alkyl, lead alkyl or aluminum alkyl, to obtain an additional increase in the activity.

### EXPERIMENTAL PART

Comparative measurements on a tube reactor using raffinate II as feed

A previously freshly activated metathesis catalyst (10% by weight of Re₂O₇ on a gamma-Al₂O₃ support) was installed in a tube reactor (metathesis reactor). An adsorbent to be tested could also be introduced before the catalyst (likewise previously freshly activated), or a similar amount of steatite spheres could be introduced for reference purposes. The ratio (gig) of adsorbent to catalyst was in the range from 2:1 to 5:1 in the experiments.

A further tube reactor (adsorber reactor) which could likewise optionally be filled with an adsorbent (>100 g) was located upstream of the metathesis reactor.

The feed used was raffinate II (a mixture comprising 1- and 2-butenes) which had previously passed through a selective hydrogenation stage so that the residual diene content was less than 15 ppm. Since only few measurements could be carried out while using one bottle of feed and the composition of the bottles was subject to small fluctuations, only measurements in the same series (i.e. using the same bottle) can be compared directly with one another, Comparisons between series of measurements are only possible when a reference measurement leads to a comparable result.

As a result of the metathesis reaction (conditions: 40° C., 35 bar), propene, 2-pentenes and 3-hexenes are formed as product from the butene mixture. The product mixture was monitored by means of on-line GC (FID) over a period of about 20 hours (cf. Figures for measurement series 1 to 5). The progressive deactivation is predominantly attributed to the presence of feed poisons which are still present in small concentrations (typically in the ppm 10 range) despite preceding purification stages. An improved action of adsorbents ideally produces both a higher initial activity and a slowing of the deactivation (i.e. a higher activity after a particular period of time t). Of the products formed, only the proportion of trans-3-hexene (formed by the self-metathesis of 1-butene) as representative compound is shown as a function of time. However, the amounts of the other products as a function of time show exactly the same effects.

It can be seen that a significant improvement in the catalyst activity or a reduction in the deactivation is obtained when using all adsorbents comprising aluminum oxide (X1-X5) after activation at temperatures above 400° C. The additional molecular sieves in the adsorber reactor can even be dispensed with entirely (measurement Q). A deactivation of this type of the molecular sieves at high temperatures (measurement S) shows no significant improvement.

| | | Preliminary | | | | Feed | Comparative |
|---|---|---|---|---|---|---|---|
| | Gas | bed | Activation of | Adsorbent | Activation of | through- | (C)/according |
| | bottle | (adsorber | preliminary | (metathesis | adsorbent | put | to the invention |
| Measurement | no. | reactor) | bed [° C.] | reactor) | [° C.] | [g/g * h] | (I) |
| A | 1 | 13X | 250 | Steatite | — | 17 | C |
| | | molecular | | | | | |
| | | sieves | | | | | |
| B | 1 | 13X | 250 | X1 | — | 17 | C |
| | | molecular | | | | | |
| | | sieves | | | | | |
| C | 1 | 13X | 250 | X1 | 550 | 17 | I |
| | | molecular | | | | | |
| | | sieves | | | | | |
| D | 1 | 13X | 250 | Y1 | 250 | 17 | C |
| | | molecular | | | | | |
| | | sieves | | | | | |
| E | 1 | 13X | 250 | X2 | 300 | 17 | C |
| | | molecular | | | | | |
| | | sieves | | | | | |
| F | 1 | 13X | 250 | X3 | 300 | 17 | C |
| | | molecular | | | | | |
| | | sieves | | | | | |
| G | 2 | 13X | 250 | Steatite | — | 25 | C |
| | | molecular | | | | | |
| | | sieves | | | | | |
| H | 2 | 13X | 250 | X1 | 550 | 25 | I |
| | | molecular | | | | | |
| | | sieves | | | | | |
| I | 2 | 13X | 250 | X4 | 550 | 25 | I |
| | | molecular | | | | | |
| | | sieves | | | | | |
| J | 3 | 13X | 250 | Steatite | — | 25 | C |
| | | molecular | | | | | |
| | | sieves | | | | | |
| K | 3 | 13X | 250 | X1 | 550 | 25 | I |
| | | molecular | | | | | |
| | | sieves | | | | | |
| L | 3 | 13X | 250 | X5 | 550 | 25 | I |
| | | molecular | | | | | |
| | | sieves | | | | | |
| M | 4 | 13X | 250 | Steatite | — | 25 | C |
| | | molecular | | | | | |
| | | sieves | | | | | |
| N | 4 | 13X | 250 | X2 | 350 | 25 | C |
| | | molecular | | | | | |
| | | sieves | | | | | |
| O | 4 | 13X | 250 | X2 | 450 | 25 | I |
| | | molecular | | | | | |
| | | sieves | | | | | |
| P | 4 | 13X | 250 | X2 | 550 | 25 | I |
| | | molecular | | | | | |
| | | sieves | | | | | |
| Q | 4 | X2 | 550 | Steatite | — | 25 | I |
| R | 5 | 13X molecular | 250 | Steatite | — | 25 | C |
| | | sieves | | | | | |
| S | 5 | 13X | 550 | Steatite | — | 25 | C |
| | | molecular | | | | | |
| | | sieves | | | | | |

X1 D10-10, 1.5 mm extrudates, BASF AG (gamma-Al₂O₃), batch comprising 900 ppm of Na
Y1 3A molecular sieves (aluminosilicate)
X2 Selexsorb CD, from Almatis (sodium aluminum silicate hydrate + gamma-Al₂O₃)
X3 Selexsorb CDO, from Almatis (sodium aluminum silicate hydrate + gamma-Al₂O₃)
X4 D10-10, 1.5 mm extrudates, BASF AG (gamma-Al₂O₃), batch comprising 100 ppm Na
X5 Alumina spheres 1.0/160, from Sasol (gamma-Al₂O₃)

1-19. (canceled)

20. A process comprising:
(a) providing a feed stream comprising a C₄ compound having at least a nonaromatic C—C double bond or C—C triple bond;
(b) contacting the feed stream with an adsorbent which has been activated at a temperature of 450 to 1000° C. comprising at least 3% by weight of aluminum oxide to remove one or more impurities from the feed stream; and
(c) contacting the feed stream from which one or more impurities have been removed with a metathesis catalyst under metathesis reaction conditions to form a compound or a mixture of compounds having a nonaromatic C—C double bond or C—C triple bond.

21. The process according to claim 20, wherein the aluminum oxide comprises a phase selected from the group consisting of gamma-Al₂O₃, delta-l₂O₃, theta-Al₂O₃, eta-Al₂O₃, and hydrated precursors thereof

22. The process according to claim 20, wherein the adsorbent comprises at least 75% by weight of aluminum oxide.

23. The process according to claim 20, wherein the activation of the adsorbent has been carried out under a reduced pressure or in an atmosphere comprising a gas selected from the group consisting of carbon dioxide, air, nitrogen, natural gas and mixtures thereof.

24. The process according to claim 20, wherein prior to the activation of the adsorbent, the adsorbent is brought into contact with an inorganic mineral acid and the mineral acid is removed.

25. The process according to claim 20, wherein the adsorbent further comprises a catalytically active amount of component selected from the group consisting of alkali metal compounds, alkaline earth metal compounds, lanthanide compounds, and zinc compounds.

26. The process according to claim 20, wherein the adsorbent has a surface area of at least 50 m²/g and a pore volume of at least 0.3 ml/g.

27. The process according to claim 20, wherein the feed stream comprises a butene and ethylene.

28. The process according to claim 27, wherein the feed stream further comprises butane.

29. The process according to claim 20, wherein the feed stream comprises 1-butene, 2-butene or a mixture thereof and the compound or mixture of compounds formed under metathesis reaction conditions comprises propene, 3-hexene, ethylene, 2-pentene or a mixture thereof.

30. The process according to claim 20, wherein the process is carried out continuously by passing the feed stream continuously through a guard bed comprising the adsorbent; and subsequently passing the feed stream continuously through a catalyst bed which comprises the metathesis catalyst.

31. The process according to claim 20, wherein providing the feed stream comprises subjecting a hydrocarbon starting material to a steam cracking or FCC process and taking off a C₄-olefin mixture comprising 1-butene, 2-butene and more than 1000 ppm by weight of butadienes from a stream formed in the cracking process; and subjecting the C₄-olefin mixture to selective hydrogenation or extractive distillation or a combination thereof to provide the feed stream, such that the feed stream has a 1,3-butadiene content of not more than 1000 ppm by weight.

32. The process according to claim 31, wherein the hydrocarbon starting material comprises naphtha.

33. The process according to claim 20, wherein providing the feed stream comprises subjecting a butane-containing starting material to dehydrogenation and subsequent purification to form a C₄-olefin mixture comprising 1-butene, 2-butene and more than 1000 ppm by weight of butadienes; and subjecting the C₄-olefin mixture to selective hydrogenation or extractive distillation or a combination thereof to provide the feed stream, such that the feed stream has a 1,3-butadiene content of not more than 1000 ppm by weight.

34. The process according to claim 20, wherein the feed stream and the adsorbent are contacted with each other at a temperature of 0 to 150° C. and a pressure of 2 to 100 bar.

35. The process according to claim 20, wherein the activation of the adsorbent is carried out over a guard bed which has previously been brought into contact with the feed stream for 1 hour to 4 months.

36. The process according to claim 20, wherein the metathesis catalyst comprises at least one compound comprising at least one element selected from the group consisting of Re, W and Mo.

37. The process according to claim 20, wherein the metathesis catalyst comprises rhenium oxide on aluminum oxide, and the metathesis reaction is carried out in the liquid phase at a temperature of 0 to 120° C.

38. The process according to claim 20, wherein the one or more impurities comprises a compound selected from the group consisting of water, alcohols, ethers, ketones, aldehydes, acids, acid derivatives, amines, nitriles, thiols, acetylenes and dienes.
